(19) United States
(12) Patent Application Publication
(10) Pub. No.: US 2009/0187173 A1
(43) Pub. Date: Jul. 23, 2009

(54) SYSTEM AND METHOD FOR RESHAPING AN EYE FEATURE

(76) Inventor: David Muller, Boston, MA (US)

Correspondence Address: NIXON PEABODY, LLP, 401 9TH STREET, NW, SUITE 900, WASHINGTON, DC, 20004-2128, US

(21) Appl. No.: 12/018,473

(22) Filed: Jan. 23, 2008

(51) Int. Cl. A61F 9/08; A61B 18/18

(52) U.S. Cl. 606/5; 606/11

(57) ABSTRACT

A system for applying therapy to an eye includes an energy source and a conducting element operably connected to the energy source and configured to direct energy from the energy source to an application end of the conducting element. The application end includes an eye contact portion configured to apply the energy to an eye feature and provides a reshaping mold to reshape the eye feature as the eye feature responds to the application of the energy. The eye contact portion may have a concave curvature and may be positioned in direct contact with the eye feature. In addition, the eye feature may be the cornea of the eye. In a particular embodiment, the energy source is an electrical energy source, the conducting element comprises an outer electrode and an inner electrode separated by a gap, and the eye contact portion is positioned on the inner electrode.

### BACKGROUND OF THE INVENTION

1. Field of the Invention

The invention pertains generally to the field of keratoplasty and, more particularly, to a system and method for applying additional reshaping forces to the cornea during thermokeratoplasty.

2. Description of Related Art

A variety of eye disorders, such as myopia, keratoconus, and hyperopia, involve abnormal shaping of the cornea. Keratoplasty reshapes the cornea to correct such disorders. For example, with myopia, the shape of the cornea causes the refractive power of an eye to be too great and images to be focused in front of the retina. Flattening aspects of the cornea's shape through keratoplasty decreases the refractive power of an eye with myopia and causes the image to be properly focused at the retina.

Invasive surgical procedures, such as laser-assisted in-situ keratonomileusis (LASIK), may be employed to reshape the cornea. However, such surgical procedures typically require a healing period after surgery. Furthermore, such surgical procedures may involve complications, such as dry eye syndrome caused by the severing of corneal nerves.

Thermokeratoplasty, on the other hand, is a noninvasive procedure that may be used to correct the vision of persons who have disorders associated with abnormal shaping of the cornea, such as myopia, keratoconus, and hyperopia. Thermokeratoplasty, for example, may be performed by applying electrical energy in the microwave or radio frequency (RF) band. In particular, microwave thermokeratoplasty may employ a near field microwave applicator to apply energy to the cornea and raise the corneal temperature. At about 60° C., the collagen fibers in the cornea shrink. The onset of shrinkage is rapid, and stresses resulting from this shrinkage reshape the corneal surface. Thus, application of energy in circular, ring-shaped patterns around the pupil generates heat that may cause aspects of the cornea to flatten and improve vision in the eye. Although thermokeratoplasty has been identified as a technique for eye therapy, there is a need for a practical and improved system for applying thermokeratoplasty, particularly in a clinical setting.

### SUMMARY OF THE INVENTION

It has been discovered that as energy is applied to the cornea during thermokeratoplasty, the corneal structure experiences changes that make the cornea susceptible to deformation by the application of additional mechanical forces. In other words, the cornea exhibits momentary plastic behavior. As such, embodiments according to aspects of the present invention provide a system and method for applying reshaping forces during thermokeratoplasty. In particular, embodiments provide a system and method for employing a shaped applicator that forms a mold against which the cornea can be further reshaped. Advantageously, embodiments provide an improved system and method for achieving a desired reshaping of a cornea by additionally applying external molding forces while the corneal fibers responds to the application of energy.

Accordingly, an embodiment of the present invention provides a system for applying therapy to an eye, including an energy source and a conducting element operably connected to the energy source. The conducting element is configured to direct energy from the energy source to an application end of the conducting element. The application end includes an eye contact portion configured to apply the energy to an eye feature. The application end also provides a reshaping mold to reshape the eye feature as the eye feature responds to the application of the energy. The eye contact portion may have a concave curvature and may be positioned in direct contact with the eye feature. The application end may be integral with the conducting element or it may be a detachable and/or disposable element that is attached to the conducting element. In addition, the eye feature may be the cornea of the eye.

In a particular embodiment, the energy source is an electrical energy source, and the conducting element includes an outer electrode and an inner electrode separated by a gap, where the eye contact portion is positioned on the inner electrode. When the conducting element is applied to the corneal surface for example, the area of the cornea at the periphery of the inner electrode is subject to an energy pattern with substantially the same shape and dimension as the gap between the two microwave conductors. As such, the energy pattern applied to the cornea is formed outside the reshaping mold provided by the inner electrode. This causes the eye contact portion of the inner electrode to be advantageously positioned with respect to the plasticity exhibited by the cornea.

Embodiments may include a positioning system configured to receive the conducting element and position the conducting element relative to a surface of the eye. The positioning system allows the eye contact portion to apply a molding pressure to the eye while the energy from the energy source is delivered to the application end of the conducting element. In a particular embodiment, the positioning system includes a vacuum ring which receives the conducting element and is adapted to create a vacuum connection with the eye and to position the conducting element relative to the eye.

Embodiments may also employ a cooling delivery system that delivers pulses of coolant to the eye to help prevent heat-related damage. In a particular embodiment, the operation of the coolant system minimizes the amount of fluid between the eye contact portion and the eye feature to enable more accurate application of the molding forces.

Correspondingly, a method for applying therapy to an eye determines a target area for eye therapy according to at least one dimension of a conducting element. The method applies a molding pressure to the area of the eye by positioning an eye contact portion of the conducting element into engagement with the target area of the eye, and also applies energy to the target area via the conducting element. The molding pressure is determined by a shape of the eye contact area. The energy causes the targeted area of the eye to conform to a new shape, where the new shape is determined at least partially by the molding pressure.

As described previously, the application of energy may be applied to cause a flattening of the cornea to improve particular types of eye conditions, such as myopia. It is understood that the embodiments described herein are not limited to causing a flattening of the cornea. In general, embodiments may achieve any type of reshaping of any structural aspect or feature of the eye. For example, rather than flattening the cornea, embodiments may apply a shaped applicator to cause the cornea to be steepened or reshaped in an asymmetric fashion.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 illustrates a cross-sectional view of an embodiment employing an electrical energy conducting element in combination with a shaped applicator to apply external molding forces to the cornea according to aspects of the present invention.

FIG. 2 illustrates another cross-sectional view of the embodiment of FIG. 1.

FIG. 3A illustrates a high resolution image of a cornea after energy has been applied.

FIG. 3B illustrates another high resolution images of the cornea of FIG. 2A.

FIG. 3C illustrates a histology image of the cornea of FIG. 2A.

FIG. 3D illustrates another histology image of the cornea of FIG. 2A.

FIG. 4 illustrates a perspective view of an energy conducting element that has an inner electrode with a contoured surface for applying external molding forces to the cornea according to aspects of the present invention.

FIG. 5A illustrates a cross-sectional view of another embodiment employing an electrical energy conducting element in combination with a shaped applicator to apply external molding forces to the cornea according to aspects of the present invention.

FIG. 5B illustrates a cross-sectional view of yet another embodiment employing an electrical energy conducting element in combination with a shaped applicator to apply external molding forces to the cornea according to aspects of the present invention.

FIG. 6 illustrates a cross-sectional view of a further embodiment employing an electrical energy conducting element in combination with a shaped applicator to apply external molding forces to the cornea according to aspects of the present invention.

FIG. 7 illustrates another embodiment employing an optical energy conducting element in combination with a shaped applicator to apply external molding forces to the cornea according to aspects of the present invention.

### DETAILED DESCRIPTION

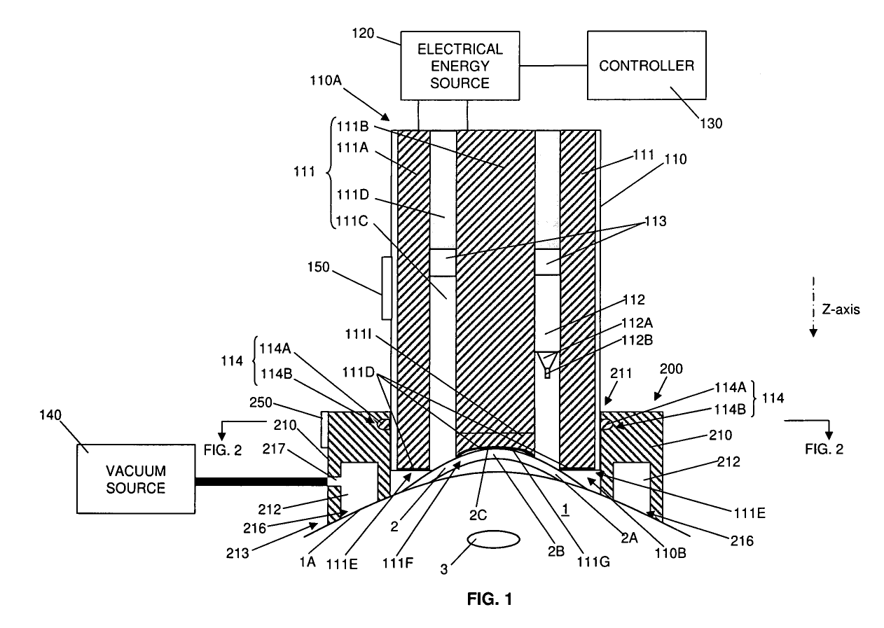

Referring to the cross-sectional view of FIG. 1, a system for applying energy to a cornea 2 of an eye 1 to achieve corrective reshaping of the cornea is illustrated. In particular, FIG. 1 shows an applicator 110 that includes an energy conducting element 111. The energy conducting element 111 extends through the applicator 110 from a proximal end 110A to a distal end 110B. An electrical energy source 120 is operably connected to the energy conducting element 111 at the distal end 110B, for example, via conventional conducting cables. The electrical energy source 120 may include a microwave oscillator for generating microwave energy. For example, the oscillator may operate at a microwave frequency range of 500 MHz to 3000 MHz, and more specifically at a frequency of around 915 MHz which provides safe use of the energy conducting element 111. Although embodiments described herein may employ microwave frequencies, it is contemplated that any frequency, e.g., including microwave, radio-frequency (RF), etc., may be employed. For example, embodiments may employ radiation having, but not limited to, a frequency between 10 MHz and 300 GHz.

Operation of the energy source 120 causes energy to be conducted through the energy conducting element 111 to the distal end 110B. As such, the applicator 110 may be employed to apply energy to the cornea 2 of the eye 1 which is positioned at the distal end 110B. As shown further in FIG. 1, the distal end 110B is positioned over the cornea 2 by a positioning system 200. In general, the positioning system 200 provides support for the applicator 110 so that the energy conducting element 111 can be operated to deliver energy to targeted areas of the cornea 2. The positioning system 200 includes an attachment element 210 which receives the applicator 110. Meanwhile, the attachment element 210 can be fixed to a portion of the eye surface 1A, such as the area surrounding the cornea 2. The attachment element 210 situates the applicator 110 in a stable position for delivering energy to the cornea 2. When applying energy to the cornea 2 with an energy conducting element 111 as shown in FIG. 1, the energy conducting element 111 may be centered, for example, over the pupil 3, which is generally coincident with a center portion 2C of the cornea 2.

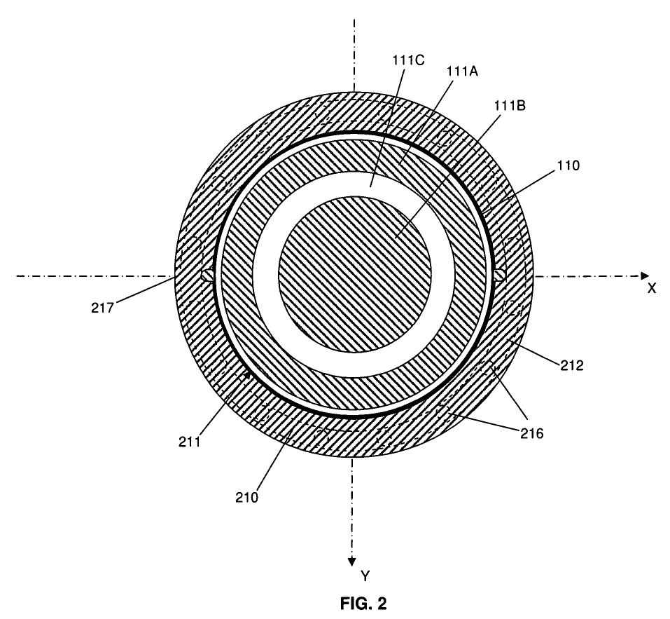

As shown in FIG. 1, the attachment element 210 of the positioning system 200 may have a substantially annular structure defining a central passageway 211 through which the applicator housing 110 can be received and the cornea 2 can be accessed. In some embodiments, for example, an outer diameter of the annular structure may range from approximately 18 mm to 23 mm while an inner diameter may range from approximately 11 mm to 15 mm to accommodate aspects of the eye 1 and the cornea 2. The attachment element 210 may be attached to portions of the eye surface 1A by creating a vacuum connection with the eye surface 1A. As such, the attachment element 210 of FIG. 1 acts as a vacuum ring that includes an interior channel 212 which is operably connected to a vacuum source 140 via connection port 217. The attachment element 210 also includes a plurality of openings 216 which open the interior channel 212 to the eye surface IA. The attachment element 210 may be formed from a biocompatible material such as a titanium alloy or the like. FIG. 2 illustrates a cross-sectional view of the attachment element 210, including the central passageway 211, the interior channel 212, the plurality of openings 216, and the connection port 217.

When the openings 216 are positioned in contact with the eye surface 1A and the vacuum source 140 is activated to create a near vacuum or low pressure within the interior channel 212, the openings 216 operate to suction the attachment element 210 and the eye surface 1A together. To promote sufficient suction between the eye surface 1A and the attachment element 210, the bottom surface 213 of the attachment element 210 may be contoured to fit the shape of the eye more closely. In one example, the vacuum source 140 may be a syringe, but the vacuum source 140 may be any manual or automated system that creates the appropriate amount of suction between the attachment element 210 and the eye surface 1A. Although the attachment element 210 can be stably attached to the eye surface 1A, the attachment element 210 can be detached by removing the vacuum source 140 and equalizing the pressure in the interior channel 212 with the exterior environment.

Once the applicator 110 is positioned by the positioning system 200, the energy conducting element 111 can deliver energy to targeted areas of collagen fibers in a mid-depth region 2B of the cornea 2 to shrink the collagen fibers according to a predetermined pattern and reshape the cornea 2 in a desired manner, thereby improving vision through the eye 1. For example, a contribution to the corneal reshaping comes from the contraction of the collagen fibrils found in the upper third of the corneal stroma, lying approximately 75-150 microns below the corneal, i.e., epithelial, surface 2A.

As further illustrated in FIG. 1, the electrical energy conducting element 111 may include two microwave conductors 111A and 111B, which extend from the proximal end 110A to the distal end 110B of the applicator 110. For example, as also illustrated in FIG. 2, the conductor 111A may be a substantially cylindrical outer conductor, while the conductor 111B may be a substantially cylindrical inner conductor that extends through an inner passage extending through the outer conductor 111A. With the inner passage, the outer conductor 111A has a substantially tubular shape. The inner and the outer conductors 111A and 111B may be formed, for example, of aluminum, stainless steel, brass, copper, other metals, metal-coated plastic, or any other suitable conductive material. As described in detail below, aspects of the energy conducting element 111 may be shaped or contoured at the distal end 110B to promote desired shape changes with the cornea 2.

With the concentric arrangement of conductors 111A and 111B shown in FIG. 2, a substantially annular gap 111C of a selected distance is defined between the conductors 111A and 111B. The annular gap 111C extends from the proximal end 110A to the distal end 110B. A dielectric material 111D may be used in portions of the annular gap 111C to separate the conductors 111A and 111B. The distance of the annular gap 111C between conductors 111A and 111B determines the penetration depth of microwave energy into the cornea 2 according to established microwave field theory. Thus, the microwave conducting element 111 receives, at the proximal end 110A, the electrical energy generated by the electrical energy source 120, and directs microwave energy to the distal end 111B, where the cornea 2 is positioned in accordance with the positioning system 200.

The outer diameter of the inner conductor 111B is preferably larger than the pupil 3, over which the applicator 110 is centered. In general, the outer diameter of the inner conductor 111B may be selected to achieve an appropriate change in corneal shape, i.e. keratometry, induced by the exposure to microwave energy. The outer diameter of the inner electrode 111B determines the diameter across which the refractive change to the cornea 2 is made. When the energy conducting element is applied to the corneal surface 2A, the area of the cornea 2 at the periphery of the inner electrode 111B is subject to an energy pattern with substantially the same shape and dimension as the gap 111C between the two microwave conductors 111A and 111B.

Meanwhile, the inner diameter of the outer conductor 111A may be selected to achieve a desired gap between the conductors 111A and 111B. For example, the outer diameter of the inner conductor 111B ranges from about 4 mm to about 10 mm while the inner diameter of the outer conductor 111A ranges from about 4.1 mm to about 12 mm. In some systems, the annular gap 111C may be sufficiently small, e.g., in a range of about 0.1 mm to about 2.0 mm, to minimize exposure of the endothelial layer of the cornea (posterior surface) to elevated temperatures during the application of energy by the applicator 110.

A controller 130 may be employed to selectively apply the energy any number of times according to any predetermined or calculated sequence. The controller 130, for example, may be a programmable processing device, such as a conventional desktop computer, that executes software, or stored instructions. In addition, the energy may be applied for any length of time. Furthermore, the magnitude of energy being applied may also be varied. Adjusting such parameters for the application of energy determines the extent of changes that are brought about within the cornea 2. Of course, the system attempts to limit the changes in the cornea 2 to an appropriate amount of shrinkage of collagen fibrils in a selected region. When delivering microwave energy to the cornea 2 with the applicator 110, the microwave energy may be applied with low power (of the order of 40 W) and in long pulse lengths (of the order of one second). However, other systems may apply the microwave energy in short pulses. In particular, it may be advantageous to apply the microwave energy with durations that are shorter than the thermal diffusion time in the cornea. For example, the microwave energy may be applied in pulses having a higher power in the range of 500 W to 3 KW and a pulse duration in the range of about 10 milliseconds to about one second.

Referring again to FIG. 1, at least a portion of each of the conductors 111A and 111B may be covered with an electrical insulator to minimize the concentration of electrical current in the area of contact between the corneal surface (epithelium) 2A and the conductors 111A and 111B. In some systems, the conductors 111A and 111B, or at least a portion thereof, may be coated with a material that can function both as an electrical insulator as well as a thermal conductor. A dielectric material 111D may optionally be employed along the distal end 110B of the applicator 110 to protect the cornea 2 from electrical conduction current that would otherwise flow into the cornea 2 via conductors 111A and 111B. Such current flow may cause unwanted temperature effects in the cornea 2 and interfere with achieving a maximum temperature within the collagen fibrils in a mid-depth region 2B of the cornea 2. Accordingly, the dielectric material 111D is positioned between the conductors 111A and 111B and the cornea 2. In particular, as shown in FIG. 1, the distal ends 111E and 111F of the conductors 111A and 111B include a dielectric material 111D. The dielectric material 111D may be sufficiently thin to minimize interference with microwave emissions and thick enough to prevent superficial deposition of electrical energy by flow of conduction current. For example, the dielectric material 111D may be a biocompatible material, such as Teflon®, deposited to a thickness of about 0.002 inches. In general, an interposing layer, such as the dielectric material 111D, may be employed between the conductors 111A and 111B and the cornea 2 as long as the interposing layer does not substantially interfere with the strength and penetration of the microwave radiation field in the cornea 2 and does not prevent sufficient penetration of the microwave field and generation of a desired energy pattern in the cornea 2. The dielectric material 111D may be omitted and electrical energy in the microwave or radio frequency (RF) band may be applied directly.

During operation, the distal end 110B of the applicator 110 as shown in FIG. 1 is positioned by the positioning system 200 at the corneal surface 2A. Preferably, the energy conducting element 111 makes direct contact with the corneal surface 2A. As such, the conductors 111A and 111B are positioned at the corneal surface 2A. The positioning of the conductors 111A and 111B helps ensure that the pattern of microwave energy delivered to the corneal tissue has substantially the same shape and dimension as the gap 111C between the two microwave conductors 111A and 111B.

As shown in FIG. 1, the applicator 110 may also employ a coolant system 112 that selectively applies coolant to the corneal surface to minimize heat-related damage to the corneal surface 2A during thermokeratoplasty and to determine the depth of energy delivered below the corneal surface 2A to the mid-depth region 2B. Such a coolant system enables the energy conducting element 111 to be placed into direct contact with the corneal surface 2A without causing heat-related damage. In some embodiments, the coolant may also be applied after the application of energy to preserve, or “set,” the desired shape changes by eliminating further energy-induced changes and preventing further changes to the new corneal shape. Examples of such a coolant system are described in U.S. application Ser. No. 11/898,189, filed Sep. 10, 2007, the contents of which are entirely incorporated herein by reference. For example, the coolant delivery system 112 as well as a coolant supply 113 may be positioned within the annular gap 111C. Although FIG. 1 may illustrate one coolant delivery system 112, the applicator 110 may include a plurality of coolant delivery systems 112 arranged circumferentially within the annular gap 111C. The coolant supply 113 may be an annular container that fits within the annular gap 111C, with the coolant delivery element 112 having a nozzle structure 112A extending downwardly from the coolant supply 113 and an opening 112B directed toward the distal end 110B. The coolant may be a liquid cryogen, such as tetrafluorothane. Alternatively, the coolant may be a cool gas, such as nitrogen gas, e.g., blowoff from a liquid nitrogen source.

In some embodiments, the coolant system 112 is operated, for example, with the controller 130 to deliver pulses of coolant in combination with the delivery of energy to the cornea 2. Advantageously, applying the coolant in the form of pulses can help prevent the creation of a fluid layer between the conductors 111A and 111B and the corneal surface 2A. In particular, the short pulses of coolant may evaporate from the corneal surface 2A or may be removed, for example, by a vacuum (not shown) before the application of the microwave energy. Rather than creating an annular energy pattern according to the dimensions of the conductors 111A and 111B, the presence of such a fluid layer may disadvantageously cause a less desirable circle-shaped microwave energy pattern in the cornea 2 with a diameter less than that of the inner conductor 111B. Therefore, to achieve a desired microwave pattern in some embodiments, a flow of coolant or a cooling layer does not exist over the corneal surface 2A during the application of energy to the cornea 2. To further minimize the presence of a fluid layer, as described previously, the coolant may actually be a cool gas, rather than a liquid coolant.

Of course, in other embodiments, a flow of coolant or a cooling layer can be employed, but such a layer or flow is generally controlled to promote the application of a predictable microwave pattern. Additionally or alternatively, heat sinks may also be employed to direct heat away from the corneal surface 2A and reduce the temperature at the surface 2A.

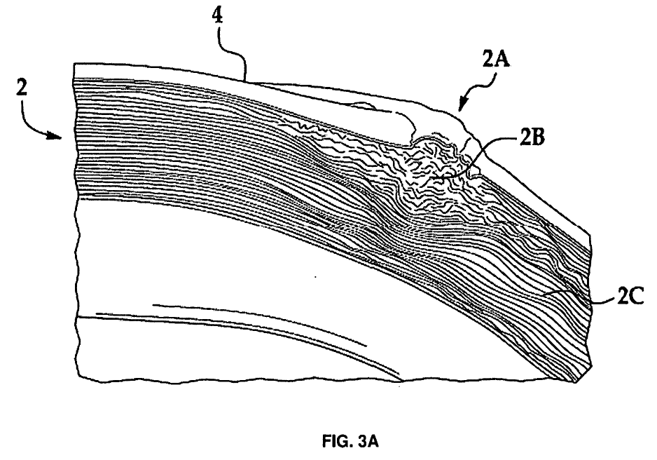

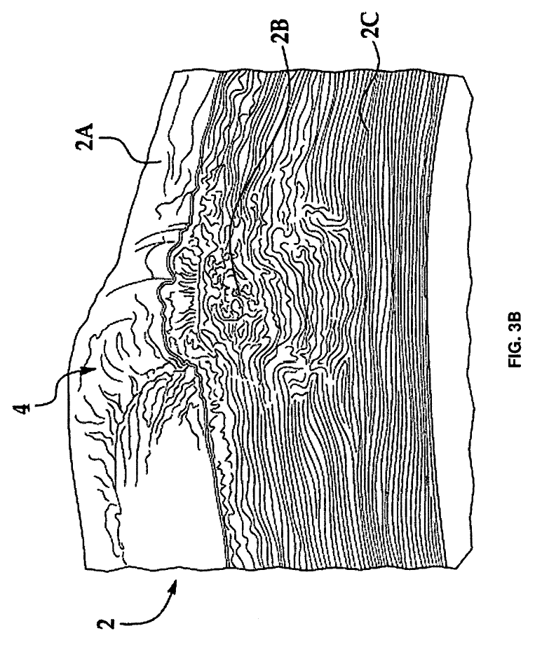

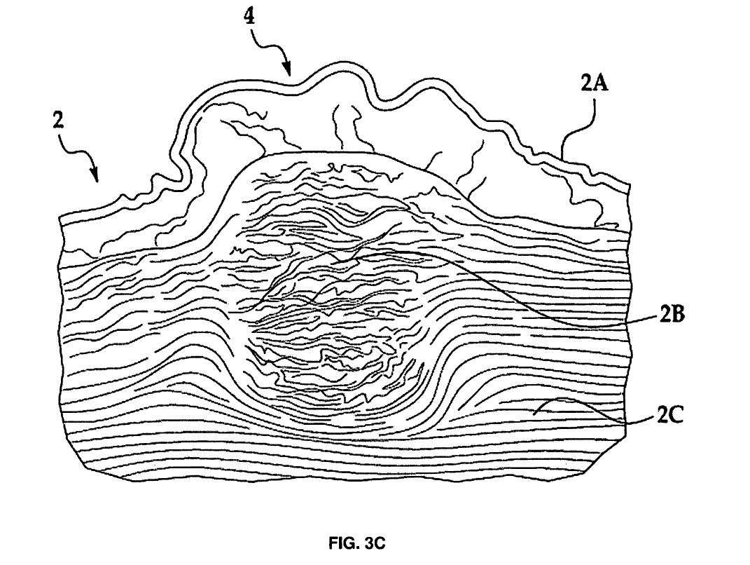

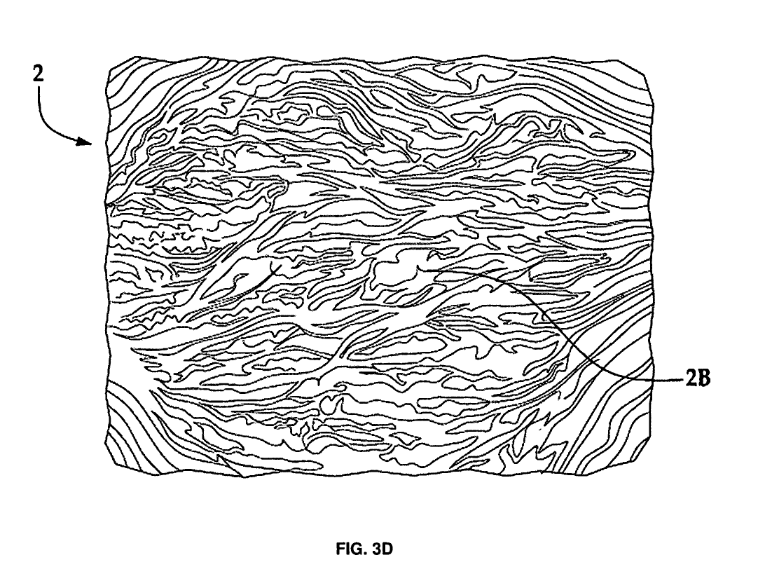

FIGS. 3A-D illustrate an example of the effect of applying energy to corneal tissue with a system for applying energy, such as the system illustrated in FIG. 1. In particular, FIGS. 3A and 3B illustrate high resolution images of the cornea 2 after energy has been applied. As FIGS. 3A and 3B show, a lesion 4 extends from the corneal surface 3A to a mid-depth region 3B in the corneal stroma 2D. The lesion 4 is the result of changes in corneal structure induced by the application of energy as described above. These changes in structure result in an overall reshaping of the cornea 2. It is noted that the application of energy, however, has not resulted in any heat-related damage to the corneal tissue.

As further illustrated in FIGS. 3A and 3B, the changes in corneal structure are localized and limited to an area and a depth specifically determined by an applicator as described above. FIGS. 3C and 3D illustrate histology images in which the tissue shown in FIGS. 3A and 3B has been stained to highlight the structural changes induced by the energy. In particular, the difference between the structure of collagen fibrils in the mid-depth region 2B where energy has penetrated and the structure of collagen fibrils outside the region 2B is clearly visible. Thus, the collagen fibrils outside the region 2B remain generally unaffected by the application of energy, while the collagen fibrils inside the region 2B have been rearranged and form new bonds to create completely different structures. In sum, the corneal areas experience a thermal transition to achieve a new state.

It has been discovered that as the corneal fibrils experience this thermal transition, there is a period in which the cornea also exhibits a plastic behavior, where the corneal structure experiences changes that make the cornea more susceptible to deformation by the application of additional mechanical forces. Therefore, embodiments employ a shaped applicator 110 that applies an external molding pressure to the cornea 2, while the cornea 2 is reshaped with the shrinkage of corneal fibers in response to the application of energy during thermokeratoplasty.

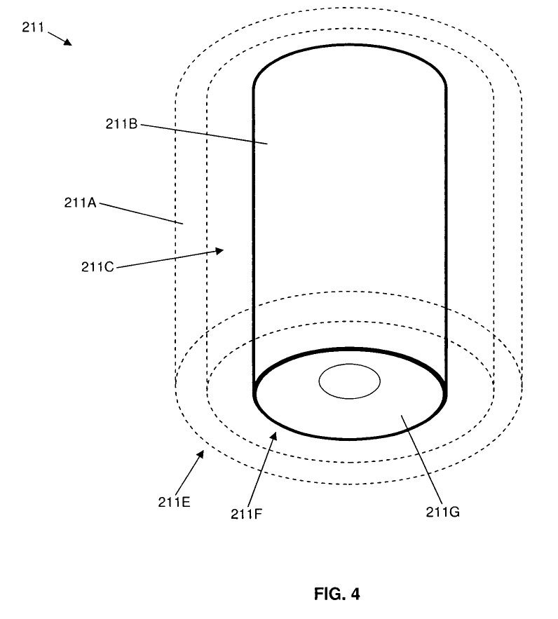

Accordingly, as illustrated in FIG. 1, the distal end 110B of the applicator 110 is configured to apply a molding pressure, or compression, to the corneal surface 2A and reshape the cornea 2 as the corneal structure experiences the state transition associated with the application of energy. As described previously, the energy conducting element 111 makes direct contact with the corneal surface 2A. FIG. 1 shows that the distal end 111F of the inner electrode 111B is in contact with the corneal surface 2A. Specifically, as also shown in FIG. 4, the distal end 111F has a surface 111G which is concave and forms a mold over the center portion 2C of the cornea 2. FIG. 4 highlights the inner electrode 111B according to aspects of the present invention.

As described previously, when the conducting element is applied to the corneal surface, the area of the cornea at the periphery of the inner electrode is subject to an energy pattern with substantially the same shape and dimension as the gap between the two microwave conductors. As such, the energy pattern applied to the cornea is formed outside the reshaping mold provided by the inner electrode 111B. In other words, the areas of the cornea 2 that are subject to plastic deformation caused by the inner electrode 111B are located inside the areas of the cornea 2 that receive the energy according to the gap 111C between the outer electrode 111A and the inner electrode 111B. This causes the surface 111G to be advantageously positioned with respect to the plasticity exhibited by the cornea 2.

During operation of the energy conducting element 111, the surface 111G is placed into contact with the portion 2C of the cornea 2 to apply molding pressures to the cornea 2. The amount of pressure applied by the surface 111G to an area of the corneal portion 2C depends on the shape of the surface 111G. For a given area of contact between the surface 111G and the portion 2C of the cornea, a greater pressure is exerted by the corresponding section of the surface 111G as the section extends farther against the cornea 2. As such, a particular shape for the surface 111G is selected to apply the desired molding profile.

While the surface 111G may be integrally formed on the inner conductor 111B, the surface 111G may also be formed on an application end piece 111I, as shown in FIG. 1, that can be removably attached to the rest of the inner conductor 111B at the distal end 110B. As such, the surface 111G can be removed or changed. Advantageously, a variety of shapes for the surface 111G may be employed with a single inner conductor 111B by interchanging different end pieces 111I, each having a different corresponding surface 111G. In other words, instead of using a separate inner conductor 111B for each shape, a single energy conducting element 111 can accommodate different reshaping requirements. Furthermore, the end pieces 111I may be disposable after a single use to promote hygienic use of the applicator 110. The end piece 111I may be removably attached with the rest of the inner conductor 111B using any conductive coupling that still permits energy to be sufficiently conducted to the cornea 2. For example, the end piece 111I may be received via threaded engagement, snap connection, other mechanical interlocking, or the like.

The curvature of the surface 111G may approximate a desired corneal shape that will improve vision through the cornea 2. However, the actual curvature of the surface 111G may need to be greater than the desired curvature of the cornea 2, as the cornea 2 may not be completely plastic and may exhibit some elasticity that can reverse some of the deformation caused by the molding pressures. Moreover, as a flattening of the cornea 2 may be desired, the curvature of the surface 111G may also include flat portions.

While the energy may be applied to cause a flattening of the cornea to improve particular types of eye conditions, such as myopia. It is understood that the embodiments described herein are not limited to causing a flattening of the cornea. Accordingly, embodiments in general may employ a shaped surface 111G that achieves any type of reshaping. For example, rather than flattening the cornea, embodiments may apply a shaped applicator to cause the cornea to be steepened or reshaped in an asymmetric fashion.

As described previously, some embodiments of the present invention do not maintain a fluid layer or a fluid flow between the energy conducting element 111 and the corneal surface 2A, thereby achieving a more predictable microwave pattern. Advantageously, in such embodiments, the molding pressures applied via the surface 111G are also more predictable as the contact between the surface 111G and the corneal area 2C is not affected by an intervening fluid layer or fluid flow.

As also described previously, the positioning system 200 places the distal end 110B of the applicator in a stable position over the cornea 2. As a result, the positioning system 200 may be employed to ensure that the surface 111G remains in contact with the corneal surface 2A and corresponding molding pressures are applied to the center portion 2C while energy is delivered via the energy conducting element 111. For example, as shown in FIG. 1, a coupling system 114 may be employed to couple the applicator 110 to the attachment element 210 of the positioning system 200. Once the applicator 110 is fully received into the attachment 210, the coupling system 114 prevents the applicator 110 from moving relative to the attachment element 210 along the Z-axis shown in FIG. 1. Thus, in combination with the attachment element 210, the energy conducting element 111, more particularly the surface 111G of the inner electrode 111B, can maintain its position against the corneal surface 2A and apply molding pressures to the center portion 2C of the cornea 2.

The coupling system 114 may include coupling elements 114A, such as tab-like structures, on the applicator 110 which are received into cavities 114B on the attachment element 210. As such, the coupling elements 114A may snap into engagement with the cavities 114B. The coupling elements 114A may be retractable to facilitate removal of the applicator 110 from the attachment element 210. For example, the coupling elements 114A may be rounded structures that extend from the applicator 110 on springs, e.g. coil or leaf springs (not shown). Additionally, the position of the coupling elements 114A along the Z-direction on the applicator 110 may be adjustable to ensure appropriate positioning of the applicator 110 with respect to the eye surface 2A and to provide the appropriate amount of molding pressure to the center portion 2C of the cornea 2.

It is understood, however, that the coupling system 114 may employ other techniques, e.g. mechanically interlocking or engaging structures, for coupling the applicator 110 to the attachment element 210. For example, the central passageway 211 of the attachment element 210 may have a threaded wall which receives the applicator 110 in threaded engagement. In such an embodiment, the applicator 110 may be screwed into the attachment element 210. The applicator can then be rotated about the Z-axis and moved laterally along the Z-axis to a desired position relative to the cornea 2.

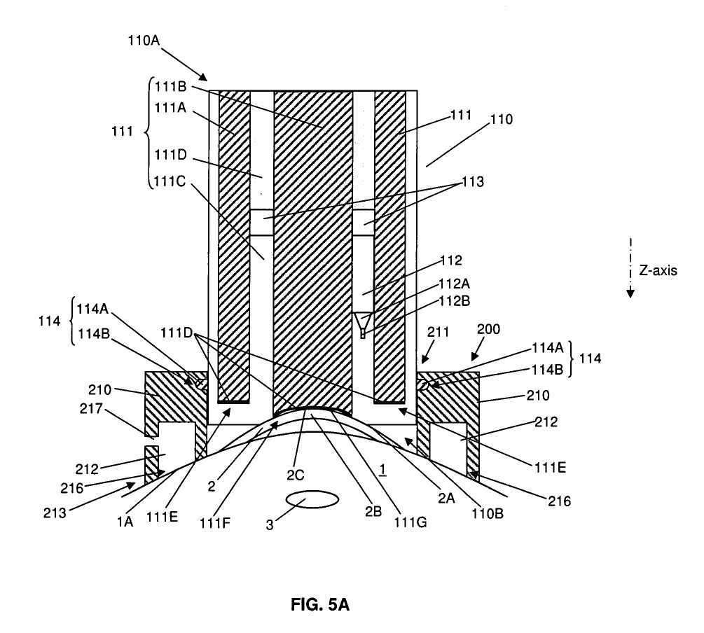

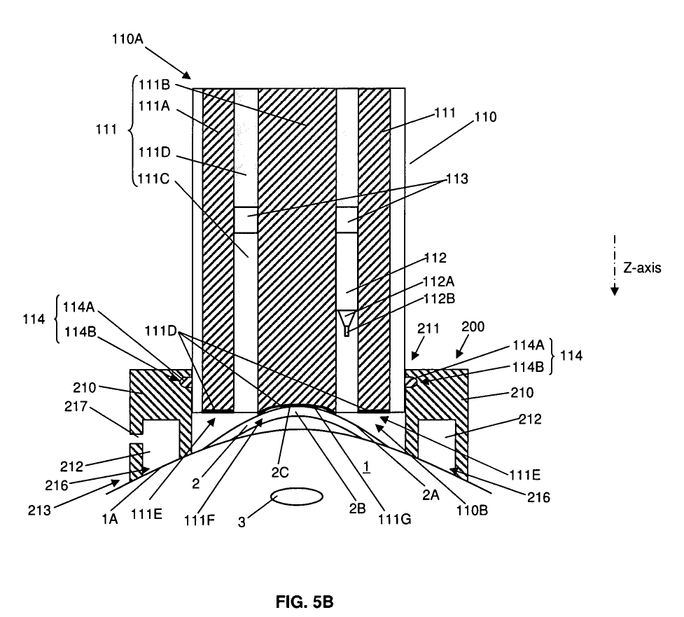

Although the distal end 111E of the outer electrode 111A shown in FIG. 1 extends past the distal end 111F of the inner electrode 111B, the position of the inner distal end 111F along the Z-axis is not limited to such a recessed position with respect to the outer distal end 111E. As shown in FIG. 5A, the inner distal end 111F may extend past the outer distal end 111E. Meanwhile, as shown in FIG. 5B, the inner distal end 111F and the outer distal end 111E extend to substantially the same position along the Z-axis.

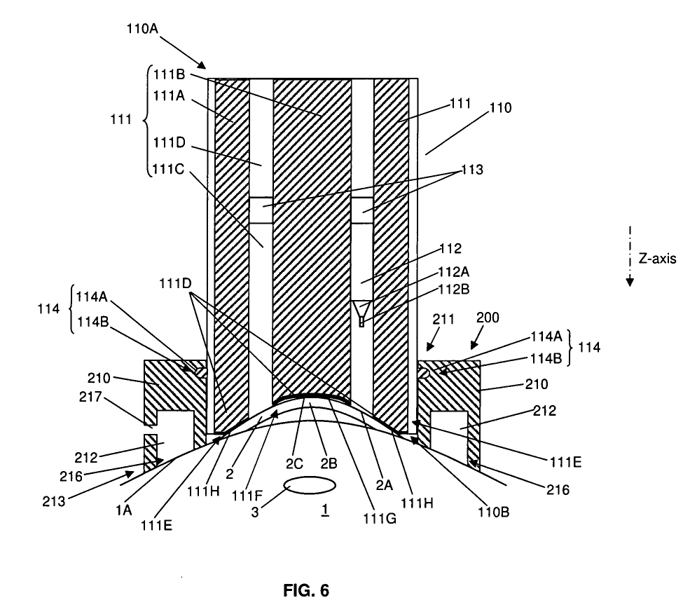

Additionally, as FIG. 6 illustrates, the distal end 111E of the outer electrode 111A may have a surface 111H that makes contact with the eye surface 1A. In some cases, the outer electrode 111A makes contact with the corneal surface 2A. Furthermore, the surface 111H may have a contoured surface that corresponds with the shape of the eye 1 where the surface 111H makes contact.

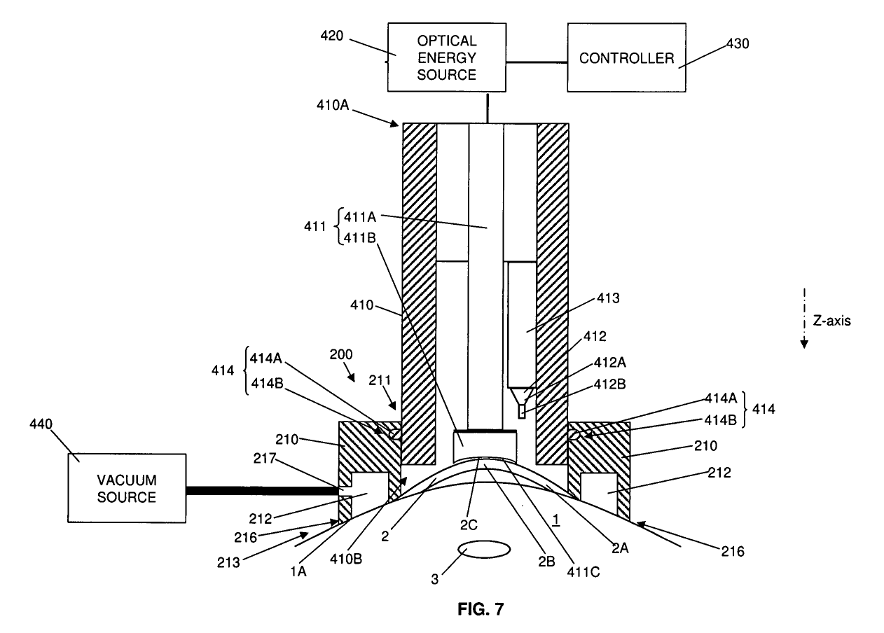

Although the energy conducting element 111 in the previous embodiments conduct electrical energy to the cornea 2, it is also contemplated that other systems may be employed to apply energy to cause reshaping of the cornea. As shown in FIG. 7, another embodiment employs an applicator 410 that includes an optical energy conducting element 411. The optical energy conducting element 411 is operably connected to an optical energy source 420, for example, via conventional optical fiber. The optical energy source 420 may include a laser, a light emitting diode, or the like. The optical energy conducting element 411 extends to the distal end 410B from the proximal end 410A, where it is operably connected with the optical source 420. The optical energy conducting element 411 includes an optical fiber 411A. Thus, the optical fiber 411A receives optical energy from the optical energy source 420 at the proximal end 410A and directs the optical energy to the distal end 410B, where the cornea 2 of an eye 1 is positioned. A controller 430 may be operably connected to the optical energy source 420 to control the delivery, e.g. timing, of the optical energy to the optical conducting element 411. The optical energy conducting element 411 irradiates the cornea 2 with the optical energy and delivers energy for appropriately shrinking collagen fibers in the mid-depth region 2B of the cornea 2. As also illustrated in FIG. 7, the optical conducting element 411 may optionally include an optical focus element 411B, such as a lens, to focus the optical energy and to determine the pattern of irradiation for the cornea 2. Like the previous embodiments, this application of energy causes the cornea 2 to experience a plastic period where the cornea 2 can be additionally reshaped by mechanical molding pressures. As such, the optical focus element 411B at the distal end 410B may include a contoured surface 411C that makes contact with the cornea surface 2A. As further illustrated by FIG. 7, the surface 411C is concave and forms a mold over a center portion 2C of the cornea 2. The contoured surface 411C may be integrally formed with the rest of the optical conducting element 411 or may be formed on a detachable end piece similar to the end piece 111I described above. For example, the optical focus element 411B which includes the surface 411C may be interchangeable with other optical focus elements 411B. Like the electrical energy conducting element 111 described previously, when the optical conducting element 411 may direct the energy to apply an energy pattern that is formed outside the reshaping mold provided by the contoured surface 411C. Thus, the areas of the cornea 2 that are subject to plastic deformation caused by the contoured surface 411C are located separately inside the areas of the cornea 2 that receive the energy according to the optical focus element 411B.

As shown in FIG. 7, the applicator 410 may also employ a coolant system 412 that selectively applies coolant to the corneal surface 2A. The coolant delivery system 412 as well as a coolant supply 413 may be positioned adjacent to the optical energy conducting element 411. The coolant system 412 may be operated, for example, with the controller 430 to deliver pulses of coolant in combination with the delivery of energy to the cornea 2. Applying the coolant in the form of pulses can help minimize the creation of a fluid layer between the optical energy conducting element 411 and the corneal surface 2A providing the advantages described previously.

As further illustrated in FIG. 7, the applicator 410 and the optical energy conducting element 411 are positioned over the cornea 2 by the positioning system 200 to deliver the optical energy to targeted areas of the cornea 2. The positioning system 200 is employed in the same manner similar to the previous embodiments. In particular, the positioning system 200 places the distal end 410B of the applicator in a stable position over the cornea 2. As a result, the positioning system 200 may be employed to ensure that the surface 411C remains in contact with the corneal surface 2A and corresponding molding pressures are applied to the center portion 2C while energy is delivered via the optical conducting element 411. For example, as described previously, a coupling system 414 may be employed to couple the applicator 110 to the attachment element 210 of the positioning system 200. The coupling system 414 may include coupling elements 414A, such as tab-like structures, on the applicator 410 which are received into cavities 414B on the attachment element 210. Once the applicator 410 is fully received into the attachment 210, the coupling system 414 prevents the applicator 110 from moving relative to the attachment element 210 along the Z-axis. Thus, in combination with the attachment element 210, the energy conducting element 411, more particularly the surface 411C of the inner electrode 411B, can maintain its position against the corneal surface 2A and apply molding pressures to center portion 2C of the cornea 2.

As described previously, the end piece 111I as shown in FIG. 1 may be disposable after a single use to promote hygienic use of the applicator 110. In general, the embodiments described herein may include disposable and replaceable components, or elements, to minimize cross-contamination and to facilitate preparation for procedures. In particular, components that are likely to come into contact with the patient's tissue and bodily fluids, such as the end piece 111I or even the entire applicator 110, are preferably discarded after a single use on the patient to minimize cross-contamination. Thus, embodiments may employ one or more use indicators which indicate whether a component of the system has been previously used. If a monitoring function determines from a use indicator that a component has been previously used, the entire system may be prevented from further operation so that the component cannot be reused and must be replaced.

For example, in the embodiment of FIG. 1, a use indicator 150 is employed to record usage data which may be read to determine whether the applicator 110 has already been used. In particular, the use indicator 150 may be a radio frequency identification (RFID) device, or similar data storage device, which contains usage data. The controller 130 may wirelessly read and write usage data to the RFID 150. For example, if the applicator 110 has not yet been used, an indicator field in the RFID device 150 may contain a null value. Before the controller 130 delivers energy from the energy source 120 to the energy conducting element 111, it reads the field in the RFID device 150. If the field contains a null value, this indicates to the controller 130 that the applicator 110 has not been used previously and that further operation of the applicator 110 is permitted. At this point, the controller 130 writes a value, such as a unique identifier associated with the controller 130, to the field in the RFID device 150 to indicate that the applicator 110 has been used. When a controller 130 later reads the field in the RFID device 150, the non-null value indicates to the controller 130 that the applicator 110 has been used previously, and the controller will not permit further operation of the applicator 110. Of course, the usage data written to the RFID device 150 may contain any characters or values, or combination thereof, to indicate whether the component has been previously used.

In another example, where the applicator 110 and the positioning system 200 in the embodiment of FIG. 1 are separate components, use indicators 150 and 250 may be employed respectively to indicate whether the application 110 or the positioning system 200 has been used previously. Similar to the use indicator 150 described previously, the use indicator 250, for example positioned on the attachment element 210, may be an RFID device which the controller 130 accesses wirelessly to read or write usage data. Before permitting operation of the applicator 110, the controller 130 reads the use indicators 150 and 250. If the controller 130 determines from the use indicators 150 and 250 that the applicator 110 and/or the positioning system 200 has already been used, the controller 130 does not proceed and does not permit further operation of the applicator 110. When the applicator 110 and the positioning system 200 are used, the controller 130 writes usage data to both use indicators 150 and 250 indicating that the two components have been used.

While various embodiments in accordance with the present invention have been shown and described, it is understood that the invention is not limited thereto. The present invention may be changed, modified and further applied by those skilled in the art. For example, although the applicators 210 and 410 in the examples above are separate elements received into the positioning system 200, the applicator 210 or 410 and the positioning system 200 may be combined to form a more integrated device. Additionally, although the attachment element 210 in the embodiments above may be a vacuum device which is auctioned to the eye surface, it is contemplated that other types of attachment elements may be employed. For instance, the attachment element may be fixed to other portions of the head. Therefore, this invention is not limited to the detail shown and described previously, but also includes all such changes and modifications.

While various embodiments in accordance with the present invention have been shown and described, it is understood that the invention is not limited thereto. The present invention may be changed, modified and further applied by those skilled in the art. Therefore, this invention is not limited to the detail shown and described previously, but also includes all such changes and modifications.

It is also understood that the Figures provided in the present application are merely illustrative and serve to provide a clear understanding of the concepts described herein. The Figures are not “to scale” and do not limit embodiments to the specific configurations and spatial relationships illustrated therein. In addition, the elements shown in each Figure may omit some features of the illustrated embodiment for simplicity, but such omissions are not intended to limit the embodiment.

What is claimed is:

1. A device for applying therapy to an eye, the system comprising:
an energy source; and
a conducting element operably connected to the energy source and configured to direct energy from the energy source to an application end of the conducting element, the application end including an eye contact portion configured to apply the energy to an eye feature and providing a reshaping mold to reshape the eye feature as the eye feature responds to the application of the energy.

2. The system according to claim 1, wherein the energy is applied to a surface area of the eye outside the reshaping mold.

3. The system according to claim 1, wherein the eye contact portion has a concave curvature.

4. The system according to claim 1, wherein the eye contact portion is positioned in direct contact with the eye feature.

5. The system according to claim 1, wherein the application end is interchangeable with another application end.

6. The system according to claim 5, wherein the other application end has a different eye contact portion.

7. The system according to claim 1, wherein a dielectric material is applied to the eye contact portion.

8. The system according to claim 1, wherein the energy source is an electrical energy source, and the conducting element comprises an outer electrode and an inner electrode separated by a gap, and the eye contact portion is positioned on the inner electrode.

9. The system according to claim 8, wherein the eye contact portion on the inner electrode extends beyond an end of the outer electrode.

10. The system according to claim 8, wherein the eye contact portion on the inner electrode is recessed in a channel defined by the outer electrode.

11. The system according to claim 8, wherein the eye contact portion on the inner electrode extends to a distance substantially even with an end of the outer electrode.

12. The system according to claim 1, wherein the energy source is an optical energy source, and the conducting element is an optical conducting element.

13. The system according to claim 1, further comprising:
a use indicator associated with the energy conducting element; and
a controller connected to the energy conducting element, the controller being operable to deliver energy generated by the energy source to the energy conducting element to direct the energy to the eye, only when the use indicator indicates that the energy conducting element has not been previously used.

14. The system according to claim 13, wherein the use indicator is a radio frequency identification (RFID) device including data readable by the controller, the data indicating whether the energy conducting element has been previously used.

15. The system according to claim 1, further comprising a positioning system configured to receive the conducting element and position the conducting element relative to a surface of the eye, allowing the eye contact portion to apply a molding pressure to the eye while the energy from the energy source is delivered to the application end of the conducting element.

16. The system according to claim 15, wherein the positioning system comprises a vacuum ring receiving the conducting element, the vacuum ring being adapted to create a vacuum connection with the eye and to position the conducting element relative to the eye.

17. The system according to claim 1, further comprising a cooling delivery system being operable to deliver pulses of coolant to the eye.

18. The system according to claim 1, wherein the eye feature is a cornea.

19. A method for applying therapy to an eye, the method comprising the steps of:
determining an area of an eye with at least one dimension of a conducting element;
applying a molding pressure to the area of the eye by positioning an eye contact portion of the conducting element into engagement with the area of the eye, the molding pressure being determined by a shape of the eye contact area; and
applying energy to the area of the eye via the conducting element, the energy causing the area of the eye to conform to a new shape, the new shape being determined at least partially by the molding pressure.

20. The method according to claim 19, wherein the step of applying energy to the area of the eye includes applying energy to a surface area of the eye outside the reshaping mold.

21. The method according to claim 19, wherein the shape of the eye contact portion has a concave curvature.

22. The method according to claim 19, further comprising attaching a detachable application element to the conducting element, the application element including the eye contact portion.

23. The method according to claim 19, further comprising disposing of the detachable application element after a single use.

24. The method according to claim 19, wherein the step of applying a molding pressure comprises placing the eye contact portion in direct contact with the area of the eye.

25. The method according to claim 19, wherein the conducting element conducts electrical energy and includes an outer electrode and an inner electrode separated by a gap, the area of the eye is determined by at least one dimension of the outer electrode, and the eye contact portion is positioned on the inner electrode.

26. The method according to claim 19, wherein the conducting element conducts optical energy.

27. The method according to claim 19, further comprising applying pulses of coolant to the eye via a cooling delivery system.

28. The method according to claim 19, wherein the area of the eye includes a part of a cornea.

29. The method according to claim 19, further comprising, before the step of applying energy, determining from a use indicator whether the energy conducting element has not been previously used.

30. The method according to claim 29, further comprising preventing operation of the energy conducting device if the use indicator indicates that the energy conducting element has been previously used.

31. The method according to claim 29, further comprising reading data from the use indicator, wherein the use indicator is a radio frequency identification (RFID) device.

32. The method according to claim 31, further comprising writing data to the use indicator, the data indicating whether the energy conducting element has been previously used.

33. The method according to claim 19, wherein the step of positioning an eye contact portion comprises:
attaching a positioning system to a surface of the eye; and
coupling the conducting element to the positioning system, the positioning system holding the conducting element in a position relative to the area of the eye and allowing the eye contact portion to apply a molding pressure to the eye while the energy is applied to the area of the eye via the conducting element.

34. The method according to claim 33, wherein the positioning system comprises a vacuum ring receiving the conducting element, the vacuum ring being adapted to create a vacuum connection with the eye and to position the conducting element relative to the eye.
